(19) United States
(12) Patent Application Publication
(10) Pub. No.: US 2026/0256729 A1
(43) Pub. Date: Sep. 3, 2026

(54) CARBAMOYL PHENYLALANINOL COMPOUNDS AS TAAR1 AGONISTS

(71) Applicant: AXSOME THERAPEUTICS, INC., New York, NY (US)

(72) Inventors: Hema GURSAHANI, Palo Alto, CA (US); Wayne MACFADDEN, Palo Alto, CA (US)

(21) Appl. No.: 18/870,855

(22) PCT Filed: Jun. 2, 2023
(86) PCT No.: PCT/US2023/067833

§ 371 Date: Dec. 2, 2024

Related U.S. Application Data
(60) Provisional application No. 63/348,767, filed on Jun. 3, 2022.

(51) Int. Cl. A61K 31/27; A61K 31/165; A61K 31/17; A61K 31/18; A61K 31/255; A61K 31/325; A61P 25/00

(52) U.S. Cl. CPC A61K 31/27; A61K 31/165; A61K 31/17; A61K 31/18; A61K 31/255; A61K 31/325; A61P 25/00

(57) ABSTRACT

The present invention relates to methods for treating trace amine-associated receptor 1 (TAAR1)-associated disorders in a subject in need thereof, comprising administering to the subject a therapeutically effective amount of carbamoyl phenylalaninol compounds that are TAAR1 agonists. The compounds may further be serotonin 1A receptor (5-HT_{1A}) agonists. TAAR1-associated disorders include sleep disorders, neurological disorders, metabolic disorders, and immune disorders.

### STATEMENT OF PRIORITY

This application claims the benefit of U.S. Provisional Application Ser. No. 63/348,767, filed Jun. 3, 2022, the entire contents of which are incorporated by reference herein.

### FIELD OF THE INVENTION

The present invention relates to methods for treating trace amine-associated receptor 1 (TAAR1)-associated disorders in a subject in need thereof, comprising administering to the subject a therapeutically effective amount of carbamoyl phenylalaninol compounds that are TAAR1 agonists. The compounds may further be serotonin 1A receptor (5-HT_{1A}) agonists. TAAR1-associated disorders include sleep disorders, neurological disorders, metabolic disorders, and immune disorders.

### BACKGROUND

Trace amine-associated receptor 1 (TAAR1) is an intracellular amine-activated Gₛ-coupled and G_{q}-coupled G protein-coupled receptor (GPCR) that is primarily expressed in several peripheral organs and cells (e.g., the stomach, small intestine, duodenum, and white blood cells), astrocytes, and in the intracellular milieu within the presynaptic plasma membrane (i.e., axon terminal) of monoamine neurons in the central nervous system (CNS). TAAR1 plays a significant role in regulating neurotransmission in dopamine, norepinephrine, and serotonin neurons in the CNS; it also affects immune system and neuroimmune system function through different mechanisms. TAAR1 is a high-affinity receptor for amphetamine, methamphetamine, dopamine, and trace amines which mediates some of their cellular effects in monoamine neurons within the central nervous system. The primary endogenous ligands of the human TAAR1 (hTAAR1) receptor, by rank order of potency, are: tyramine>β-phenethylamine>dopamine=octopamine.

TAAR1 is widely expressed across the mammalian brain, particularly in limbic and monoaminergic areas, allegedly involved in mood, attention, memory, fear, and addiction. There is evidence to suggest that TAAR1 may be a promising therapeutic target for the treatment of schizophrenia, psychosis in Parkinson's disease, substance use disorders, and glucose homeostasis, diabetes, metabolic syndrome, and obesity.

There is a need in the art for novel TAAR1 agonists for treatment of TAAR1-associated disorders.

### SUMMARY OF EMBODIMENTS OF THE INVENTION

The present invention is based in part on the identification of solriamfetol ((R)-2-amino-3-phenylpropyl carbamate; SUNOSI™) as a TAAR1 agonist as well as a serotonin 1A receptor (5-HT_{1A}) agonist.

Thus, one aspect of the invention relates to a method for treating a TAAR1-associated disorder in a subject in need thereof, comprising administering to the subject a therapeutically effective amount of a carbamoyl phenylalaninol compound that activates TAAR1, thereby treating the TAAR1-associated disorder. In some embodiments, the carbamoyl phenylalaninol compound also activates the 5-HT_{1A} receptor.

The TAAR1-associated disorder may be a sleep disorder, a neurological disorder, a metabolic disorder, or an immune disorder.

In some embodiments, the carbamoyl phenylalaninol compound is a compound of Formula I:

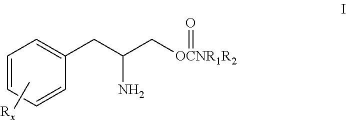

or a pharmaceutically acceptable salt or ester thereof;
wherein R is a member selected from the group consisting of alkyl of 1 to 8 carbon atoms, halogen, alkoxy of 1 to 3 carbon atoms, nitro, hydroxy, trifluoromethyl, and thioalkoxy of 1 to 3 carbon atoms;
x is an integer of 0 to 3, with the proviso that R may be the same or different when x is 2 or 3;
R₁ and R₂ are independently selected from the group consisting of hydrogen, alkyl of 1 to 8 carbon atoms, aryl, arylalkyl, cycloalkyl of 3 to 7 carbon atoms; or
R₁ and R₂ can be joined to form a 5- to 7-membered heterocycle that is unsubstituted or substituted with one or more alkyl groups or aryl groups, wherein the heterocycle can comprise 1 to 2 nitrogen atoms and 0 to 1 oxygen atom, wherein the nitrogen atoms are not directly connected with each other or with the oxygen atom.

In some embodiments, the carbamoyl phenylalaninol compound is a compound of Formula II:

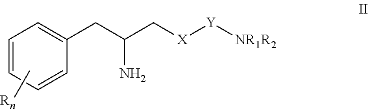

or a pharmaceutically acceptable salt thereof, wherein:
X is CH₂, O, NH, or S;
Y is C═O, C═S, or SO₂;
R is optionally substituted C₁₋₈ alkyl, halogen, optionally substituted C₁₋₄ alkoxy, cyano, hydroxy, optionally substituted trifluoromethyl, or C₁₋₄ thioalkoxy;
n is 0, 1, 2, or 3, with the proviso that R may be the same or different when x is 2 or 3; and
R₁ and R₂ can be the same or different and are independently selected from the group consisting of hydrogen, optionally substituted C₁₋₈ alkyl, optionally substituted amide, optionally substituted aryl, optionally substituted heteroaryl, optionally substituted arylalkyl, and optionally substituted C₃₋₇ cycloalkyl;
or R₁ and R₂ can be joined to form a 5- to 7-membered heterocycle optionally substituted with alkyl or aryl groups, wherein the cyclic compound can comprise 1 to 2 nitrogen atoms and 0 to 1 oxygen atom, wherein the nitrogen atoms are not directly connected with each other or with the oxygen atom;
wherein when Y is C═O, X is not O.

The present invention is explained in greater detail in the drawings herein and the specification set forth below.

### BRIEF DESCRIPTION OF THE DRAWINGS

FIG. 1 shows that solriamfetol is a dopamine and norepinephrine reuptake inhibitor that activates hTAAR1 and 5-HT_{1A} in vitro at clinically relevant plasma concentrations. 5-HT_{1A}, serotonin 1A receptor; hDAT, human dopamine transporter; hNET, human norepinephrine transporter; hTAAR1, human trace amine-associated receptor 1.

FIGS. 2A and 2B shows that solriamfetol inhibits firing frequency of ventral tegmental area (VTA) neurons in a D2 receptor-sensitive manner. Solriamfetol inhibits firing by VTA neurons in a dose-dependent manner, similar to TAAR1 agonist RO5256390. aCSF, artificial cerebrospinal fluid.

FIGS. 3A and 3B show that a reduction in firing frequency by solriamfetol or TAAR1 agonist RO5256390 was antagonized by pre-treatment with the D2 receptor antagonist sulpiride. aCSF, artificial cerebrospinal fluid.

FIG. 4A-4C show solriamfetol inhibits hyperlocomotion in dopamine transporter deficient (DAT^{-/-}) mice. FIG. 4A, Solriamfetol does not increase locomotor activity in wild type mice, unlike a stimulant (amphetamine, Amph). Shown is the post-injection response (0-90 minutes). FIG. 4B, Solriamfetol reduces hyperlocomotion in DAT^{-/-} mice, similar to a stimulant (Amph). Shown is the post-injection response (0-90 minutes). FIG. 4C, RO5166017, an established TAAR1 agonist, reduced hyperlocomotion in DAT^{-/-} mice. *P<0.05 vs vehicle; **P<0.01 vs vehicle.

### DETAILED DESCRIPTION OF EMBODIMENTS OF THE INVENTION

The present invention now will be described hereinafter with reference to the accompanying drawings and examples, in which embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a composition comprises components A, B and C, it is specifically intended that any of A, B or C, or a combination thereof, can be omitted and disclaimed singularly or in any combination.

### Definitions

As used herein, “a,” “an,” or “the” can mean one or more than one. For example, “a” cell can mean a single cell or a multiplicity of cells.

Also as used herein, “and/or” refers to and encompasses any and all possible combinations of one or more of the associated listed items, as well as the lack of combinations when interpreted in the alternative (“or”).

The term “about,” as used herein when referring to a measurable value such as an amount of dose (e.g., an amount of a compound) and the like, is meant to encompass variations of ±10%, ±5%, ±1%, ±0.5%, or even ±0.1% of the specified amount.

The terms “comprise,” “comprises,” and “comprising” as used herein, specify the presence of the stated features, integers, steps, operations, elements, and/or components, but do not preclude the presence or addition of one or more other features, integers, steps, operations, elements, components, and/or groups thereof.

As used herein, the transitional phrase “consisting essentially of” means that the scope of a claim is to be interpreted to encompass the specified materials or steps recited in the claim and those that do not materially affect the basic and novel characteristic(s) of the claimed invention. Thus, the term “consisting essentially of” when used in a claim or the description of this invention is not intended to be interpreted to be equivalent to “comprising.”

As used herein, the terms “increase,” “increases,” “increased,” “increasing,” and similar terms indicate an elevation of at least about 25%, 50%, 75%, 100%, 150%, 200%, 300%, 400%, 500% or more.

As used herein, the terms “reduce,” “reduces,” “reduced,” “reduction,” and similar terms mean a decrease of at least about 5%, 10%, 15%, 20%, 25%, 35%, 50%, 75%, 80%, 85%, 90%, 95%, 97% or more. In particular embodiments, the reduction results in no or essentially no (i.e., an insignificant amount, e.g., less than about 10% or even 5%) detectable activity or amount.

“Effective amount” as used herein refers to an amount of a compound, composition and/or formulation of the invention that is sufficient to produce a desired effect, which can be a therapeutic and/or beneficial effect. The effective amount will vary with the age, general condition of the subject, the severity of the condition being treated, the particular agent administered, the duration of the treatment, the nature of any concurrent treatment, the pharmaceutically acceptable carrier used, and like factors within the knowledge and expertise of those skilled in the art. As appropriate, an “effective amount” in any individual case can be determined by one of skill in the art by reference to the pertinent texts and literature and/or by using routine experimentation.

By the term “treat,” “treating,” or “treatment of” (and grammatical variations thereof) it is meant that the severity of the subject's condition is reduced, at least partially improved or ameliorated and/or that some alleviation, mitigation or decrease in at least one clinical sign or symptom is achieved and/or there is a delay in the progression of the disease or disorder. With respect to a TAAR1-associated disorder, the term refers to a decrease in one or more signs or symptoms of the TAAR1-associated disorder described herein in a subject by at least 5%, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%.

A “therapeutically effective” amount as used herein is an amount that is sufficient to treat (as defined herein) the subject. Those skilled in the art will appreciate that the therapeutic effects need not be complete or curative, as long as some benefit is provided to the subject.

The terms “prevent,” “preventing,” and “prevention” (and grammatical variations thereof) refer to prevention and/or delay of the onset of a disease, disorder and/or a clinical symptom(s) in a subject and/or a reduction in the severity of the onset of the disease, disorder and/or clinical symptom(s) relative to what would occur in the absence of the methods of the invention. The prevention can be complete, e.g., the total absence of the disease, disorder and/or clinical symptom(s). The prevention can also be partial, such that the occurrence of the disease, disorder and/or clinical symptom(s) in the subject and/or the severity of onset is less than what would occur in the absence of the present invention. With respect to a TAAR1-associated disorder, the term refers to, e.g., preventing the TAAR1-associated disorder from occurring if the treatment is administered prior to the onset of the disorder.

A “prevention effective” amount as used herein is an amount that is sufficient to prevent and/or delay the onset of a disease, disorder and/or clinical symptoms in a subject and/or to reduce and/or delay the severity of the onset of a disease, disorder and/or clinical symptoms in a subject relative to what would occur in the absence of the methods of the invention. Those skilled in the art will appreciate that the level of prevention need not be complete, as long as some benefit is provided to the subject.

A “subject” of the invention includes any animal that has or is susceptible to a TAAR1-associated disorder or is in need of treatment of a TAAR1-associated disorder. Such a subject is generally a mammalian subject (e.g., a laboratory animal such as a rat, mouse, guinea pig, rabbit, primate, etc.), a farm or commercial animal (e.g., a cow, horse, goat, donkey, sheep, etc.), or a domestic animal (e.g., cat, dog, ferret, etc.). In particular embodiments, the subject is a primate subject, a non-human primate subject (e.g., a chimpanzee, baboon, monkey, gorilla, etc.) or a human. Subjects include males and/or females of any age, including neonates, juveniles, adolescents, adults, and geriatric subjects.

A “subject in need” of the methods of the invention can be a subject known to have, suspected of having, or having an increased risk of developing a TAAR1-associated disorder.

As used herein the term “TAAR1-associated disorder” refers to any disorder that is known to be caused by or have at least one symptom that results from modulation of TAAR1, e.g., inhibition of TAAR1 activity or modulation of TAAR1 ligands. Additionally, “TAAR1-associated disorder” includes any disorder in which administration of a TAAR1 agonist to a subject results in the treatment of one or more symptoms of the disorder in the subject.

The term “pharmaceutically acceptable salts or esters” shall mean non-toxic salts or esters of the compounds employed in this invention which are generally prepared by reacting the free acid with a suitable organic or inorganic base or the free base with a suitable organic or inorganic acid. Examples of such salts include, but are not limited to, acetate, benzenesulfonate, benzoate, bicarbonate, bisulfate, bitartrate, borate, bromide, calcium, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynapthoate, iodide, isothionate, lactate, lactobionate, laurate, malate, maleate, mandelate, mesylate, methylbromide, methylnitrate, methylsulfate, mucate, napsylate, nitrate, oleate, oxalate, pamoate, palmitate, panthothenate, phosphate/diphosphate, polygalacturonate, potassium, salicylate, sodium, stearate, subacetate, succinate, tannate, tartrate, teoclate, tosylate, triethiodide, and valerate.

As used herein the term “concomitant administration” or “combination administration” of a compound, therapeutic agent or known drug with a compound of the present invention means administration of a known medication or drug and, in addition, the one or more compounds of the invention at such time that both the known drug and the compound will have a therapeutic effect. In some cases, this therapeutic effect will be synergistic. Such concomitant administration can involve concurrent (i.e., at the same time), prior, or subsequent administration of the known drug with respect to the administration of a compound of the present invention. A person of skill in the art, would have no difficulty determining the appropriate timing, sequence, and dosages of administration for particular drugs and compounds of the present invention.

In addition, in some embodiments, the compounds of this invention will be used, either alone or in combination with each other or in combination with one or more other therapeutic medications as described above, or their salts or esters, for manufacturing a medicament for the purpose of providing treatment for a TAAR1-associated disorder to a patient or subject in need thereof.

The term “alkyl” denotes a straight or branched hydrocarbon chain containing 1-12 carbon atoms, e.g., 1-8 (C₁₋₈), 1-6 (C₁₋₆), or 1-4 (C₁₋₄) carbon atoms. Examples of alkyl groups include methyl, ethyl, propyl, isopropyl, butyl, isobutyl, tert-butyl, and the like.

By “substituted alkyl” is meant an alkyl in which an atom of the alkyl is substituted with, for example, a carbon, nitrogen, sulfur, oxygen, silicon, or halogen atom, or alternatively a nitrogen, sulfur, oxygen, or halogen atom. The term encompasses substituents on alkyl, alkenyl, alkynyl, and cycloalkyl groups.

Examples of substituents that can be attached to any atom of the alkyl group in a “substituted alkyl” include cyclyl groups, heterocyclyl groups; aryl groups, heteroaryl groups, amino groups, amido groups, nitro groups, cyano groups, azide groups, hydroxy groups, alkoxy groups, acyloxy groups, thioalkoxy groups, acyl thioalkoxy groups, halogen groups, sulfonate groups, sulfonamide groups, ester groups, carboxylic acids, oxygen (e.g., a carbonyl group), and sulfur (e.g., a thiocarbonyl group). Substituents also include any chemical functional group that imparts improved water-solubility to the molecule (e.g., carboxylic acid, carboxylic ester, carboxamido, morpholino, piperazinyl, imidazolyl, thiomorpholino, or tetrazolyl groups; both unsubstituted and substituted).

The terms “halo” and “halogen” refer to any radical of fluorine, chlorine, bromine, or iodine.

The term “alkoxy” denotes an oxygen linked to an alkyl or substituted alkyl as defined above.

The term “thioalkoxy” denotes a sulfur linked to an alkyl or substituted alkyl as defined above.

The term “cycloalkyl” denotes a monocyclic saturated carbocyclic group containing 3-8 carbon atoms, e.g., 3-6 (C₃₋₆) or 3-7 (C₃₋₇) carbon atoms. Examples of cycloalkyl groups include cyclopropyl, cyclobutyl, cyclopentyl, cyclohexyl, cycloheptyl, cyclooctyl, and the like.

The term “aryl” refers to an aromatic 5-8 membered monocyclic or 8-12 membered bicyclic ring system wherein 0, 1, 2, or 3 atoms of each ring can be substituted by a substituent. The term also includes aromatic bicyclic ring systems in which a hydrogen atom has been added to one, two, or three of the ring carbons in one of the rings (e.g., a partially saturated ring). Examples of aryl groups include phenyl, naphthyl and the like.

The term “arylalkyl” denotes an aryl group linked to an alkyl or substituted alkyl as defined above.

The term “heterocycle” refers to an aromatic or nonaromatic 5-8 membered monocyclic or 8-12 membered bicyclic ring system comprising 1-3 heteroatoms if monocyclic or 1-6 heteroatoms if bicyclic, said heteroatoms selected from O, N, or S, wherein 0, 1, 2 or 3 atoms of each ring can be substituted by a substituent. The term also includes aromatic and nonaromatic bicyclic ring systems in which a hydrogen atom has been added to one, two, or three of the ring carbons in one of the rings (e.g., a partially saturated ring). Examples of heterocycle groups include pyridyl, furyl or furanyl, benzofuranyl, imidazolyl, benzimidazolyl, pyrimidinyl, thiophenyl or thienyl, benzothiophenyl, quinolinyl, isoquinolinyl, dihydroquinolinyl, dihydroisoquinolinyl, naphthyridinyl, dihydronaphthyridinyl, quinazolinyl, indolyl, indazolyl, thiazolyl, benzothiazolyl, oxazinyl, benzooxazinyl, oxazolyl, benzooxazolyl, dihydrobenzodioxinyl, and the like.

Suitable substituents for aryl and heteroaryl groups are the same as the substituents for alkyl groups.

The present invention is based in part on the identification of solriamfetol as a TAAR1 agonist as well as a serotonin 1A receptor (5-HT_{1A}) agonist. Solriamfetol activates human TAAR1 (hTAAR1) at potencies that are within the clinically relevant plasma concentration range and overlap with observed dopamine transporter/norepinephrine transporter inhibitory potencies. This is in contrast to the wake-promoting agent modafinil and the dopamine and norepinephrine reuptake inhibitor bupropion, which exhibit no hTAAR1 activity. Thus, solriamfetol and related carbamoyl phenylalaninol compounds may have unique biological activities that make them effective to treat TAAR1-associated disorders.

One aspect the present invention is directed to a method for treating a trace amine-associated receptor 1 (TAAR1)-associated disorder in a subject in need thereof, comprising administering to the subject a therapeutically effective amount of a carbamoyl phenylalaninol compound that activates TAAR1, thereby treating the TAAR1-associated disorder. In some embodiments, the carbamoyl phenylalaninol compound also activates the serotonin 1A receptor (5-HT_{1A}).

TAAR1-associated disorders, include without limitation, sleep disorders (such as narcolepsy, cataplexy, excessive daytime sleepiness (e.g., associated with idiopathic hypersomnia, multiple sclerosis, depression, or drug-associated excessive sleepiness), shift work sleep disorder, or idiopathic hypersomnia); neurological disorders (such as attention deficit disorder, attention deficit hyperactivity disorder, schizophrenia, depression, anxiety, psychosis, or addiction); metabolic disorders (such as glucose homeostasis disorders, type II diabetes, metabolic syndrome, binge eating disorders, or obesity); or immune disorders.

In some embodiments, the carbamoyl phenylalaninol compound is a compound of Formula I:

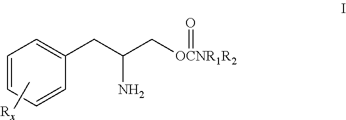

or a pharmaceutically acceptable salt or ester thereof, wherein R is a member selected from the group consisting of alkyl of 1 to 8 carbon atoms, halogen, alkoxy of 1 to 3 carbon atoms, nitro, hydroxy, trifluoromethyl, and thioalkoxy of 1 to 3 carbon atoms; x is an integer of 0 to 3, with the proviso that R may be the same or different when x is 2 or 3; R₁ and R₂ are independently selected from the group consisting of hydrogen, alkyl of 1 to 8 carbon atoms, aryl, arylalkyl, cycloalkyl of 3 to 7 carbon atoms; or R₁ and R₂ can be joined to form a 5- to 7-membered heterocycle that is unsubstituted or substituted with one or more alkyl or aryl groups, wherein the heterocycle can comprise 1 to 2 nitrogen atoms and 0 to 1 oxygen atom, wherein the nitrogen atoms are not directly connected with each other or with the oxygen atom; wherein the subject reduces body weight, thereby treating the obesity.

In some embodiments, R is a member selected from the group consisting of alkyl of 1 to 3 carbon atoms, halogen, alkoxy of 1 to 3 carbon atoms, nitro, hydroxy, and trifluoromethyl. In some embodiments of the above methods, R is a member selected from the group consisting of alkyl of 1 to 3 carbon atoms, halogen, and alkoxy of 1 to 3 carbon atoms.

In some embodiments of the above methods, R₁ and R₂ are independently selected from the group consisting of hydrogen, alkyl of 1 to 8 carbon atoms, aryl, arylalkyl, and cycloalkyl of 3 to 7 carbon atoms. In some embodiments of the above methods, R₁ and R₂ are independently selected from the group consisting of hydrogen and alkyl of 1 to 8 carbon atoms. In some embodiments of the above methods, R₁ and R₂ are independently selected from the group consisting of hydrogen and alkyl of 1 to 3 carbon atoms.

It is understood that substituents and substitution patterns on the compounds of the present invention can be selected by one of skill in the art to provide compounds that are chemically stable and that can be readily synthesized by techniques known in the art and described herein.

In one embodiment, the compound of Formula I is a compound of Formula Ia:

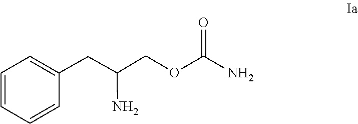 [SMILES: NC(=O)OCC(N)Cc1ccccc1]

or a pharmaceutically acceptable salt or ester thereof.

In one embodiment the compound of Formula I is the (D) enantiomer wherein R₁ and R₂ are hydrogen and x is 0 (Formula Ib).

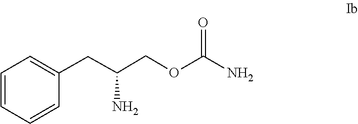 [SMILES: NC(=O)OC[C@H](N)Cc1ccccc1]

or a pharmaceutically acceptable salt or ester thereof. This compound (solriamfetol) is the (R) enantiomer, if named by structure and is therefore (R)-(beta-amino-benzenepropyl) carbamate or (R)-2-amino-3-phenylpropyl carbamate. This compound is the dextrorotary enantiomer and can therefore also be named O-carbamoyl-(D)-phenylalaninol. These names may be used interchangeably in this specification.

The present invention includes the use of isolated enantiomers of the compound of Formula I (e.g., compounds of Formula Ia or Ib). In one embodiment, a pharmaceutical composition comprising the isolated S-enantiomer of Formula I is used to provide treatment to a subject. In another embodiment, a pharmaceutical composition comprising the isolated R-enantiomer of Formula I is used to provide treatment to a subject.

The present invention also includes the use of mixtures of enantiomers of Formula I. In one aspect of the present invention, one enantiomer will predominate. An enantiomer that predominates in the mixture is one that is present in the mixture in an amount greater than any of the other enantiomers present in the mixture, e.g., in an amount greater than 50%. In one aspect, one enantiomer will predominate to the extent of 90% or to the extent of 91%, 92%, 93%, 94%, 95%, 96%, 97% or 98% or greater. In one embodiment, the enantiomer that predominates in a composition comprising a compound of Formula I is the R-enantiomer of Formula I.

The present invention provides methods of using enantiomers and enantiomeric mixtures of compounds represented by Formula I. A carbamate enantiomer of Formula I contains an asymmetric chiral carbon at the benzylic position, which is the second aliphatic carbon adjacent to the phenyl ring.

An enantiomer that is isolated is one that is substantially free of the corresponding enantiomer. Thus, an isolated enantiomer refers to a compound that is separated via separation techniques or prepared free of the corresponding enantiomer.

The term “substantially free,” as used herein, means that the compound is made up of a significantly greater proportion of one enantiomer. In preferred embodiments, the compound includes at least about 90% by weight of one enantiomer. In other embodiments of the invention, the compound includes at least about 99% by weight of one enantiomer.

The compounds of Formula I can be synthesized by methods known to the skilled artisan. The salts and esters of the compounds of Formula I can be produced by treating the compound with a suitable mineral or organic acid (HX) in suitable solvent or by other means well known to those of skill in the art.

Details of reaction schemes for synthesizing compounds of Formula I as well as representative examples on the preparation of specific compounds have been described in U.S. Pat. Nos. 5,705,640, 5,756,817, 5,955,499, 6,140,532, and 10,829,443, all incorporated herein by reference in their entirety.

In some embodiments, the carbamoyl phenylalaninol compound is a compound of Formula II:

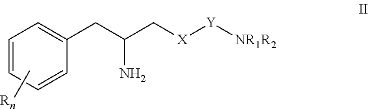

or a pharmaceutically acceptable salt thereof, wherein:
X is CH₂, O, NH, or S;
Y is C═O, C═S, or SO₂;
R is optionally substituted C₁₋₈ alkyl, halogen, optionally substituted C₁₋₄ alkoxy, cyano, hydroxy, optionally substituted trifluoromethyl, or C₁₋₄ thioalkoxy;
n is 0, 1, 2, or 3, with the proviso that R may be the same or different when x is 2 or 3; and
R₁ and R₂ can be the same or different and are independently selected from the group consisting of hydrogen, optionally substituted C₁₋₈ alkyl, optionally substituted amide, optionally substituted aryl, optionally substituted heteroaryl, optionally substituted arylalkyl, and optionally substituted C₃₋₇ cycloalkyl;
or R₁ and R₂ can be joined to form a 5- to 7-membered heterocycle optionally substituted with alkyl or aryl groups, wherein the cyclic compound can comprise 1 to 2 nitrogen atoms and 0 to 1 oxygen atom, wherein the nitrogen atoms are not directly connected with each other or with the oxygen atom;
wherein when Y is C═O, X is not O.

In some embodiments, X is CH₂, O, or NH; X is CH₂, O, or S; X is CH₂, NH, or S; X is O, NH, or S; X is CH₂ or S; X is CH₂ or NH; X is CH₂ or O; X is O or S; X is NH or S; X is O or S; X is O or NH; X is CH₂; X is O; X is NH; or X is S.

In some embodiments, Y is C═O or SO₂; Y is C═O or C═S; Y is C═S or SO₂; Y is C═O; Y is C═S; or Y is SO₂.

In some embodiments, R₁ or R₂ is C(O)NR₃R₄, wherein R₃ and R₄ are independently hydrogen, optionally substituted lower alkyl of 1 to 8 carbon atoms, optionally substituted aryl, optionally substituted arylalkyl, or optionally substituted cycloalkyl of 3 to 7 carbon atoms; or R₃ and R₄ can be joined to form a 5 to 7-membered heterocycle optionally substituted with a member selected from the group consisting of alkyl and aryl groups, wherein the heterocycle can comprise 1 to 2 nitrogen atoms and 0 to 1 oxygen atom, wherein the nitrogen atoms are not directly connected with each other or with the oxygen atom. In some embodiments, R₁ or R₂ is C(O)NH₂. See U.S. Pat. No. 10,710,958, the contents of which are incorporated by reference in their entirety,
In some embodiments, the compound of Formula II has the structure of Formula IIa:

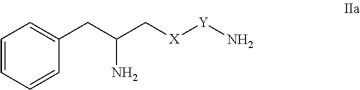

or a pharmaceutically acceptable salt thereof.

In some embodiments, the compound of Formula I has the structure of Formula III:

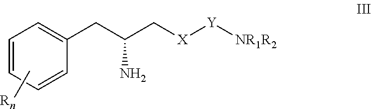

or a pharmaceutically acceptable salt thereof.

In certain embodiments, the compound of Formula III has the structure of Formula IIIa:

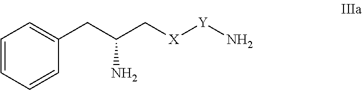

or a pharmaceutically acceptable salt thereof.

In some embodiments, the compound of Formula II has the structure of Formula IV:

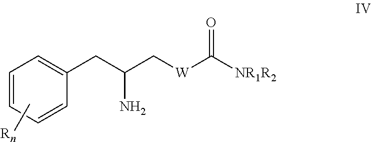

or a pharmaceutically acceptable salt thereof, wherein:
W is CH₂ or NH.

In certain embodiments, the compound of Formula IV has the structure of Formula IVa:

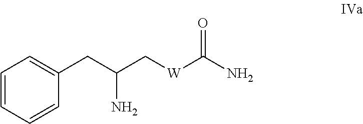

or a pharmaceutically acceptable salt thereof.

Examples of compounds within the structure of Formula IVa include without limitation, compounds 1 and 2:

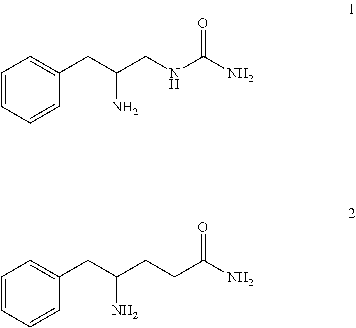 [SMILES: NC(=O)NCC(N)Cc1ccccc1 ; NC(=O)CCC(N)Cc1ccccc1]

or a pharmaceutically acceptable salt thereof.

In some embodiments, the compound of Formula IV has the structure of Formula V:

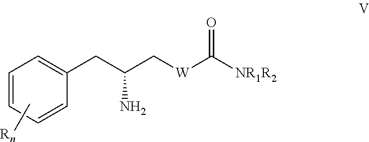

or a pharmaceutically acceptable salt thereof, wherein:
W is CH₂ or NH.

In certain embodiments, the compound of Formula V has the structure of Formula Va:

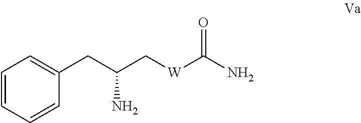

or a pharmaceutically acceptable salt thereof, wherein:
W is CH₂ or NH.

Examples of compounds within the structure of Formula Va include without limitation, compounds 3 and 4:

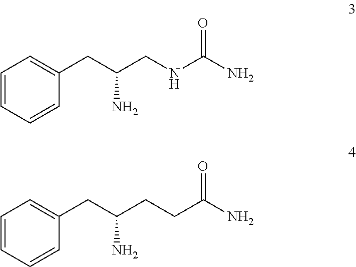 [SMILES: NC(=O)NC[C@H](N)Cc1ccccc1 ; NC(=O)CC[C@H](N)Cc1ccccc1]

or a pharmaceutically acceptable salt thereof.

In some embodiments, the compound of Formula II has the structure of Formula VI:

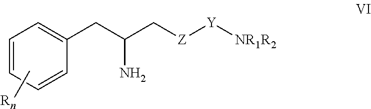

or a pharmaceutically acceptable salt thereof, wherein:
Z is O or S; and
Y is C═O, C═S, or SO₂;
wherein when Y is C═O, Z is not O.

In certain embodiments, the compound of Formula VI has the structure of Formula VIa:

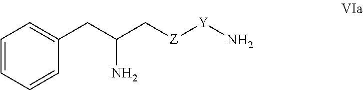

or a pharmaceutically acceptable salt thereof.

Examples of compounds within the structure of Formula VIa include without limitation, compounds 5, 6, and 7:

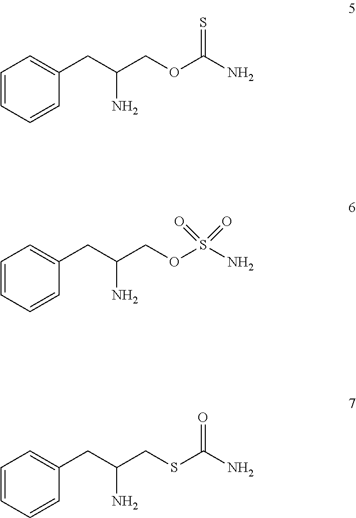 [SMILES: NC(=S)OCC(N)Cc1ccccc1 ; NC(COS(N)(=O)=O)Cc1ccccc1 ; NC(=O)SCC(N)Cc1ccccc1]

or a pharmaceutically acceptable salt thereof.

In some embodiments, the compound of Formula VI has the structure of Formula VII:

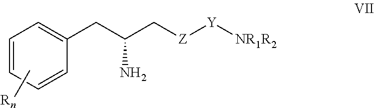

or a pharmaceutically acceptable salt thereof.

In certain embodiments, the compound of Formula VII has the structure of Formula VIIa:

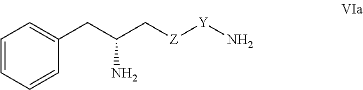

or a pharmaceutically acceptable salt thereof.

Examples of compounds within the structure of Formula VIIa include without limitation, compounds 8, 9, and 10:

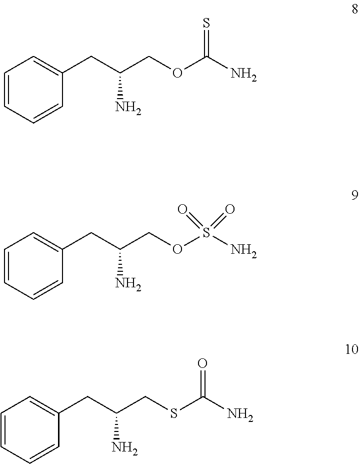 [SMILES: NC(=S)OC[C@H](N)Cc1ccccc1 ; N[C@@H](COS(N)(=O)=O)Cc1ccccc1 ; NC(=O)SC[C@H](N)Cc1ccccc1]

or a pharmaceutically acceptable salt thereof.

Details of reaction schemes for synthesizing compounds of Formulae II-VII as well as representative examples on the preparation of specific compounds have been described in U.S. Pat. No. 11,413,264, incorporated herein by reference in its entirety.

From the formulae above it is evident that some of the compounds of the invention have at least one and possibly more asymmetric carbon atoms. It is intended that the present invention include within its scope the stereochemically pure isomeric forms of the compounds as well as their racemates. Stereochemically pure isomeric forms may be obtained by the application of art known principles. Diastereoisomers may be separated by physical separation methods such as fractional crystallization and chromatographic techniques, and enantiomers may be separated from each other by the selective crystallization of the diastereomeric salts with optically active acids or bases or by chiral chromatography. Pure stereoisomers may also be prepared synthetically from appropriate stereochemically pure starting materials, or by using stereoselective reactions.

During any of the processes for preparation of the compounds of the present invention, it may be necessary and/or desirable to protect sensitive or reactive groups on any of the molecules concerned. This may be achieved by means of conventional protecting groups, such as those described in Protective Groups in Organic Chemistry, ed. J. F. W. McOmie, Plenum Press, 1973; and T. W. Greene & P. G. M. Wuts, Protective Groups in Organic Synthesis, Third Edition, John Wiley & Sons, 1999. The protecting groups may be removed at a convenient subsequent stage using methods known in the art.

The compounds, formulations and unit dosage forms provided herein can be utilized, e.g., to achieve immediate, controlled, and/or delayed release of the compound of the invention, as well as pharmaceutically acceptable salts, hydrates, isomers, including tautomers, solvates and complexes of the compound.

Suitable salts of the compound of the invention include, without limitation, acetate, adipate, alginate, aspartate, benzoate, butyrate, citrate, fumarate, glycolate, hemisulfate, heptanoate, hexanoate, hydrochloride, hydrobromide, hydroiodide, 2-hydroxyethanesulfonate, lactate, maleate, malonate, methanesulfonate, nicotinate, nitrate, oxalate, palmoate, pectinate, persulfate, hydroxynapthoate, pivalate, propionate, salicylate, succinate, sulfate, tartrate, thiocyanate, tosylate and undecanoate. Other acids, such as oxalic, while not in themselves pharmaceutically acceptable, can be employed in the preparation of salts useful as intermediates in obtaining the compound of the invention and their pharmaceutically acceptable acid addition salts. In certain embodiments, the salt is the hydrochloride salt.

Compounds of the formulae herein include those having quaternization of any basic nitrogen-containing group therein.

The discussion herein is, for simplicity, provided without reference to stereoisomerism or the addition of deuterium atoms. Those skilled in the art will appreciate that the compound of the invention can contain one or more asymmetric centers and thus occur as racemates and racemic mixtures and single optical isomers. All such isomeric and deuterated forms of these compounds are expressly included in the present invention. In some embodiments, the compound is in the form of a single stereoisomer or a mixture in which one stereoisomer predominates, e.g., by about 60%, 70%, 80%, 90%, 95%, or more.

The discussion herein is also provided without reference to polymorphs, hydrates, clathrates, solvates, inclusion compounds, isomers, or other forms of the compound. All such forms of these compounds are expressly included in the present invention.

Further, the compounds of the invention include prodrugs of the compounds that are converted to the active compound in vivo. For example, the compound can be modified to enhance cellular permeability (e.g., by esterification of polar groups) and then converted by cellular enzymes to produce the active agent. Methods of masking charged or reactive moieties as a pro-drug are known by those skilled in the art (see, e.g., P. Korgsgaard-Larsen and H. Bundgaard, A Textbook of Drug Design and Development, Reading U.K., Harwood Academic Publishers, 1991).

The term “prodrug” refers to compounds that are rapidly transformed in vivo to yield the parent compound of the above formula, for example, by hydrolysis in blood, see, e.g., T. Higuchi and V. Stella, Prodrugs as Novel delivery Systems, Vol. 14 of the A.C.S. Symposium Series and in Edward B. Roche, ed., Bioreversible Carriers in Drug Design, American Pharmaceutical Association and Pergamon Press, 1987, both of which are incorporated by reference herein. See also U.S. Pat. No. 6,680,299. Exemplary prodrugs include a prodrug that is metabolized in vivo by a subject to an active drug having an activity of the compounds as described herein, wherein the prodrug is an ester of an alcohol or carboxylic acid group, if such a group is present in the compound; an amide of an amine group or carboxylic acid group, if such groups are present in the compound; a urethane of an amine group, if such a group is present in the compound; an acetal or ketal of an alcohol group, if such a group is present in the compound; a N-Mannich base or an imine of an amine group, if such a group is present in the compound; or a Schiff base, oxime, acetal, enol ester, oxazolidine, or thiazolidine of a carbonyl group, if such a group is present in the compound, such as described, for example, in U.S. Pat. Nos. 6,680,324 and 6,680,322.

The term “pharmaceutically acceptable prodrug” (and like terms) as used herein refers to those prodrugs of the compound of the present invention which are, within the scope of sound medical judgment, suitable for use in contact with the tissues of humans and/or other animals without undue toxicity, irritation, allergic response and the like, commensurate with a reasonable risk/benefit ratio, and effective for their intended use, as well as the zwitterionic forms, where possible, of the compound of the invention.

Another aspect of the invention relates to a composition, e.g., a dosage form, comprising the compound of the invention. In some embodiments, the composition is a pharmaceutical composition comprising the compound of the invention and a pharmaceutically acceptable carrier. In some embodiments, the dosage form is an oral dosage form, e.g., a tablet or a capsule, e.g., an immediate release dosage form.

In some embodiments, the dosage form is an immediate release tablet that releases at least 85%, e.g., at least 85%, 90%, 95%, 96%, 97%, 98%, or 99%, of the compound of the invention contained therein within a period of less than 15 minutes after administration of the tablet to a subject.

Formulations of the compound of the invention, including immediate release formulations, may be processed into unit dosage forms suitable for oral administration, such as for example, filled capsules, compressed tablets or caplets, or other dosage form suitable for oral administration using conventional techniques. Immediate release dosage forms prepared as described may be adapted for oral administration, so as to attain and maintain a therapeutic level of the compound over a preselected interval. In certain embodiments, an immediate release dosage form as described herein may comprise a solid oral dosage form of any desired shape and size including round, oval, oblong cylindrical, or polygonal. In one such embodiment, the surfaces of the immediate release dosage form may be flat, round, concave, or convex. In some embodiments, the formulations may be the solid oral dosage forms described in U.S. Pat. No. 10,195,151, incorporated herein by reference in its entirety.

In particular, when the immediate release formulations are prepared as a tablet, the immediate release tablets contain a relatively large percentage and absolute amount of the compound and so are expected to improve patient compliance and convenience, by replacing the need to ingest large amounts of liquids or liquid/solid suspensions. One or more immediate release tablets as described herein can be administered, by oral ingestion, e.g., closely spaced, in order to provide a therapeutically effective dose of the compound to the subject in a relatively short period of time.

Where desired or necessary, the outer surface of an immediate release dosage form may be coated, e.g., with a color coat or with a moisture barrier layer using materials and methods known in the art.

The compound may be administered to a subject by any conventional route of administration, including, but not limited to, oral, buccal, topical, systemic (e.g., transdermal, intranasal, or by suppository), or parenteral (e.g., intramuscular, subcutaneous, or intravenous injection.) Administration of the compounds directly to the nervous system can include, for example, administration to intracerebral, intraventricular, intracerebroventricular, intrathecal, intracisternal, intraspinal or peri-spinal routes of administration by delivery via intracranial or intravertebral needles or catheters with or without pump devices. Depending on the route of administration, compounds of Formula I can be constituted into any form. For example, forms suitable for oral administration include solid forms, such as pills, gelcaps, tablets, caplets, capsules, granules, and powders (each including immediate release, timed release and sustained release formulations). Forms suitable for oral administration also include liquid forms, such as solutions, syrups, elixirs, emulsions, and suspensions. In addition, forms useful for parenteral administration include sterile solutions, emulsions and suspensions.

In certain embodiments, pharmaceutical compositions of this invention comprise one or more compounds of the invention or a salt or ester thereof without any pharmaceutical carriers or excipients. In other embodiments, pharmaceutical compositions of this invention comprise one or more compounds of the invention or a salt or ester thereof intimately admixed with a pharmaceutical carrier according to conventional pharmaceutical compounding techniques. Carriers are inert pharmaceutical excipients, including, but not limited to, binders, suspending agents, lubricants, flavorings, sweeteners, preservatives, dyes, and coatings. In preparing compositions in oral dosage form, any of the usual pharmaceutical carriers may be employed. For example, for liquid oral preparations, suitable carriers and additives include water, glycols, oils, alcohols, flavoring agents, preservatives, coloring agents and the like; for solid oral preparations, suitable carriers and additives include starches, sugars, diluents, granulating agents, lubricants, binders, disintegrating agents and the like.

Compositions can take the form of tablets, pills, capsules, semisolids, powders, sustained release formulations, solutions, suspensions, emulsions, syrups, elixirs, aerosols, or any other appropriate compositions; and comprise at least one compound of this invention, optionally in combination with at least one pharmaceutically acceptable excipient. Suitable excipients are well known to persons of ordinary skill in the art, and they, and the methods of formulating the compositions, can be found in such standard references as Alfonso AR: Remington's Pharmaceutical Sciences, 17th ed., Mack Publishing Company, Easton Pa., 1985, the disclosure of which is incorporated herein by reference in its entirety and for all purposes. Suitable liquid carriers, especially for injectable solutions, include water, aqueous saline solution, aqueous dextrose solution, and glycols.

The compounds can be provided as aqueous suspensions. Aqueous suspensions of the invention can contain a compound in admixture with excipients suitable for the manufacture of aqueous suspensions. Such excipients can include, for example, a suspending agent, such as sodium carboxymethylcellulose, methylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate).

The aqueous suspension can also contain one or more preservatives such as ethyl or n-propyl p-hydroxybenzoate, one or more coloring agents, one or more flavoring agents, and one or more sweetening agents, such as sucrose, aspartame or saccharin. Formulations can be adjusted for osmolarity.

Oil suspensions for use in the present methods can be formulated by suspending a compound in a vegetable oil, such as arachis oil, olive oil, sesame oil or coconut oil, or in a mineral oil such as liquid paraffin, or a mixture of these. The oil suspensions can contain a thickening agent, such as beeswax, hard paraffin or cetyl alcohol. Sweetening agents can be added to provide a palatable oral preparation, such as glycerol, sorbitol or sucrose. These formulations can be preserved by the addition of an antioxidant such as ascorbic acid. As an example of an injectable oil vehicle, see Minto, J. Pharmacol. Exp. Ther. 281:93 (1997). The pharmaceutical formulations of the invention can also be in the form of oil-in-water emulsions. The oily phase can be a vegetable oil or a mineral oil, as described above, or a mixture of these.

Suitable emulsifying agents include naturally occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent.

The compound of choice, alone or in combination with other suitable components can be made into aerosol formulations (i.e., they can be “nebulized”) to be administered via inhalation. Aerosol formulations can be placed into pressurized acceptable propellants, such as dichlorodifluoromethane, propane, nitrogen, and the like.

Formulations of the present invention suitable for parenteral administration, such as, for example, by intraarticular (in the joints), intravenous, intramuscular, intradermal, intraperitoneal, and subcutaneous routes, can include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. Among the acceptable vehicles and solvents that can be employed are water and Ringer's solution, an isotonic sodium chloride. In addition, sterile fixed oils can conventionally be employed as a solvent or suspending medium. For this purpose, any bland fixed oil can be employed including synthetic mono- or diglycerides. In addition, fatty acids such as oleic acid can likewise be used in the preparation of injectables. These solutions are sterile and generally free of undesirable matter.

Where the compounds are sufficiently soluble they can be dissolved directly in normal saline with or without the use of suitable organic solvents, such as propylene glycol or polyethylene glycol. Dispersions of the finely divided compounds can be made-up in aqueous starch or sodium carboxymethyl cellulose solution, or in suitable oil, such as arachis oil. These formulations can be sterilized by conventional, well-known sterilization techniques. The formulations can contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like.

The concentration of a compound in these formulations can vary widely, and will be selected primarily based on fluid volumes, viscosities, body weight, and the like, in accordance with the particular mode of administration selected and the patient's needs. For IV administration, the formulation can be a sterile injectable preparation, such as a sterile injectable aqueous or oleaginous suspension. This suspension can be formulated according to the known art using suitable dispersing or wetting agents and suspending agents. The sterile injectable preparation can also be a sterile injectable solution or suspension in a nontoxic parenterally acceptable diluent or solvent, such as a solution of 1,3-butanediol. The formulations of commends can be presented in unit-dose or multi-dose sealed containers, such as ampoules and vials. Injection solutions and suspensions can be prepared from sterile powders, granules, and tablets of the kind previously described.

A compound suitable for use in the practice of this invention can be administered orally. The amount of a compound of the present invention in the composition can vary widely depending on the type of composition, size of a unit dosage, kind of excipients, and other factors well known to those of skill in the art. In general, the final composition can comprise, for example, from 0.000001 percent by weight (% w) to 100% w of the compound, e.g., 0.00001% w to 50% w, with the remainder being the excipient or excipients.

Pharmaceutical formulations for oral administration can be formulated using pharmaceutically acceptable carriers well known in the art in dosages suitable for oral administration. Such carriers enable the pharmaceutical formulations to be formulated in unit dosage forms as tablets, pills, powder, dragees, capsules, liquids, lozenges, gels, syrups, slurries, suspensions, etc. suitable for ingestion by the patient. In other embodiments, pharmaceutical formulations for oral administration can be formulated without using any pharmaceutically acceptable carriers.

Formulations suitable for oral administration can consist of (a) liquid solutions, such as an effective amount of the pharmaceutical formulation suspended in a diluents, such as water, saline or polyethylene glycol (PEG) 400; (b) capsules, sachets or tablets, each containing a predetermined amount of the active ingredient, as liquids, solids, granules or gelatin; (c) suspensions in an appropriate liquid; and (d) suitable emulsions.

Pharmaceutical preparations for oral use can be obtained through combination of the compounds of the present invention with a solid excipient, optionally grinding a resulting mixture, and processing the mixture of granules, after adding suitable additional compounds, if desired, to obtain tablets or dragee cores. Suitable solid excipients are carbohydrate or protein fillers and include, but are not limited to sugars, including lactose, sucrose, mannitol, or sorbitol; starch from corn, wheat, rice, potato, or other plants; cellulose such as methyl cellulose, hydroxymethyl cellulose, hydroxypropylmethyl-cellulose or sodium carboxymethylcellulose; and gums including arabic and tragacanth; as well as proteins such as gelatin and collagen.

If desired, disintegrating or solubilizing agents can be added, such as cross-linked polyvinyl pyrrolidone, agar, alginic acid, or a salt thereof, such as sodium alginate. Tablet forms can include one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise the active ingredient in a flavor, e.g., sucrose, as well as pastilles comprising the active ingredient in an inert base, such as gelatin and glycerin or sucrose and acacia emulsions, gels, and the like containing, in addition to the active ingredient, carriers known in the art.

The compounds of the present invention can also be administered in the form of suppositories for rectal administration of the drug. These formulations can be prepared by mixing the drug with a suitable non-irritating excipient that is solid at ordinary temperatures but liquid at the rectal temperatures and will therefore melt in the rectum to release the drug. Such materials are cocoa butter and polyethylene glycols.

The compounds of the present invention can also be administered by intranasal, intraocular, intravaginal, and intrarectal routes including suppositories, insufflation, powders and aerosol formulations (for examples of steroid inhalants, see Rohatagi, J. Clin. Pharmacol. 35:1187 (1995); Tjwa, Ann. Allergy Asthma Immunol. 75:107 (1995)).

The compounds of the present invention can be delivered transdermally, by a topical route, formulated as applicator sticks, solutions, suspensions, emulsions, gels, creams, ointments, pastes, jellies, paints, powders, and aerosols.

Encapsulating materials can also be employed with the compounds of the present invention and the term “composition” can include the active ingredient in combination with an encapsulating material as a formulation, with or without other carriers. For example, the compounds of the present invention can also be delivered as microspheres for slow release in the body. In one embodiment, microspheres can be administered via intradermal injection of drug (e.g., mifepristone)-containing microspheres, which slowly release subcutaneously (see Rao, J. Biomater. Sci. Polym. Ed. 7:623 (1995); as biodegradable and injectable gel formulations (see, e.g., Gao, Pharm. Res. 12:857 (1995)); or, as microspheres for oral administration (see, e.g., Eyles, J. Pharm. Pharmacol. 49:669 (1997)). Both transdermal and intradermal routes afford constant delivery for weeks or months. Cachets can also be used in the delivery of the compounds of the present invention.

In another embodiment, the compounds of the present invention can be delivered by the use of liposomes which fuse with the cellular membrane or are endocytosed, i.e., by employing ligands attached to the liposome that bind to surface membrane protein receptors of the cell resulting in endocytosis. The active drug can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

By using liposomes, particularly where the liposome surface carries ligands specific for target cells, or are otherwise preferentially directed to a specific organ, one can focus the delivery of the compound into target cells in vivo (see, e.g., Al-Muhammed, J. Microencapsul. 13:293 (1996); Chonn, Curr. Opin. Biotechnol. 6:698 (1995); Ostro, Am. J. Hosp. Pharm. 46:1576 (1989)).

Active drug may also be delivered by the use of monoclonal antibodies as individual carriers to which the compound molecules are coupled. Active drug may also be coupled with soluble polymers as targetable drug carriers. Such polymers can include polyvinyl-pyrrolidone, pyran copolymer, polyhydroxy-propyl-methacrylamide-phenol, polyhydroxy-ethyl-aspartamide-phenol, or polyethyleneoxide-polylysine substituted with palmitoyl residues. Furthermore, active drug may be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacrylates and cross linked or amphipathic block copolymers of hydrogels.

In certain embodiments the compositions are in unit dosage forms such as tablets, pills, capsules, powders, granules, sterile parenteral solutions or suspensions, metered aerosol or liquid sprays, drops, ampoules, auto-injector devices or suppositories, for oral parenteral, intranasal, sublingual or rectal administration, or for administration by inhalation or insufflation.

Alternatively, the composition may be presented in a form suitable for once-weekly or once-monthly administration; for example, an insoluble salt of the active compound, such as the decanoate salt, may be adapted to provide a depot preparation for intramuscular injection.

The pharmaceutical compositions herein will contain, per dosage unit, e.g., tablet, capsule, powder, injection, teaspoonful, suppository and the like, an amount of the active ingredient necessary to deliver an effective dose as described above. For example, the pharmaceutical compositions herein can contain, per unit dosage unit, from about 10 to about 1000 mg of the active ingredient, e.g., from about 25 to about 600 mg of the active ingredient, e.g., from about 75 to about 400 mg of the active ingredient, e.g., about 25, 37.5, 50, 75, 100, 125, 150, 175, 200, 225, 250, 275, 300, 325, 350, 375, 400, 425, 450, 475, 500, 525, 550, 575, or 600 mg or more or any range therein.

In some embodiments of the present invention, compounds suitable for use in the practice of this invention will be administered either singly or concomitantly with at least one or more other compounds or therapeutic agents.

The method includes the step of administering to a patient in need of treatment or prevention an effective amount of one of the compounds disclosed herein in combination with an effective amount of one or more other compounds or therapeutic agents that have the ability to provide advantageous combined effects such as the ability to augment the effects of the compounds of the invention.

Compounds named in this invention can be present in unsalified or unesterified form, or in salified and/or esterified form, and the naming of such compounds is intended to include both the original (unsalified and unesterified) compound and its pharmaceutically acceptable salts and esters. The present invention includes pharmaceutically acceptable salt and ester forms of Formula I. More than one crystal form of an enantiomer of Formula I can exist and as such are also included in the present invention.

A pharmaceutical composition of the invention can optionally contain, in addition to a compound, at least one other therapeutic agent useful in the treatment of a TAAR1-associated disorder. For example, the compounds of the invention can be combined physically with other compounds in fixed dose combinations to simplify their administration.

Methods of formulating pharmaceutical compositions have been described in numerous publications such as Pharmaceutical Dosage Forms: Tablets. Second Edition. Revised and Expanded. Volumes 1-3, edited by Lieberman et al.; Pharmaceutical Dosage Forms: Parenteral Medications. Volumes 1-2, edited by Avis et al.; and Pharmaceutical Dosage Forms: Disperse Systems. Volumes 1-2, edited by Lieberman et al.; published by Marcel Dekker, Inc, the disclosure of each of which are herein incorporated by reference in their entireties and for all purposes.

The pharmaceutical compositions are generally formulated as sterile, substantially isotonic and in full compliance with all Good Manufacturing Practice (GMP) regulations of the U.S. Food and Drug Administration.

The present invention provides methods of providing treatment of TAAR1-associated disorders using compounds of the invention. TAAR1-associated disorders, include without limitation, sleep disorders (such as narcolepsy, cataplexy, excessive daytime sleepiness (e.g., associated with idiopathic hypersomnia, multiple sclerosis, depression, or drug-associated excessive sleepiness), shift work sleep disorder, or idiopathic hypersomnia); neurological disorders (such as attention deficit disorder, attention deficit hyperactivity disorder, schizophrenia, depression, anxiety, psychosis, or addiction); metabolic disorders (such as glucose homeostasis disorders, type II diabetes, metabolic syndrome, binge eating disorders, or obesity); or immune disorders.

Narcolepsy is a chronic neurological disorder characterized by recurring episodes of sleep or sleepiness during the day, and often disrupted nocturnal REM sleep. Symptoms of narcolepsy include abnormal sleep features, overwhelming episodes of sleep, excessive daytime sleepiness (EDS), abnormal REM sleep, hypnagogic and hypnopompic hallucinations, disturbed nocturnal sleep, cataplexy, and sleep paralysis. EDS includes daytime sleep attacks, which may occur with or without warning; persistent drowsiness, which may continue for prolonged periods of time; and “microsleeps” or fleeting moments of sleep intruding into the waking state.

Cataplexy is characterized as an abrupt and reversible decrease or loss of muscle tone most frequently elicited by emotion. It can involve a limited number of muscles or the entire voluntary musculature except the extraocular muscles and to some extent the diaphragm. Typically, the jaw sags, the head falls forward, the arms drop to the side, and/or the knees unlock, or the cataplectic human may fall completely on the ground. Attacks can be elicited by emotion, stress, fatigue, exercise or heavy meals.

Idiopathic hypersomnia is a sleep disorder that is associated with a normal or prolonged major sleep episode and excessive sleepiness consisting of prolonged (1 to 2 hour) sleep episodes of nREM sleep. Idiopathic hypersomnia can be characterized by a complaint of constant or recurrent excessive daytime sleepiness, typically with sleep episodes lasting 1 or more hours in duration. It can be enhanced in situations that allow sleepiness to become manifest, such as reading or watching television in the evening. The major sleep episode can be prolonged, lasting more than 8 hours. The capacity to arouse the subject can be normal, but some patients report great difficulty waking up and experience disorientation after awakening.

Shift work sleep disorder is a circadian rhythm sleep-wake disorder that results from the inability of some shift workers to adapt to the mismatch between the worker's sleep-wake imposed schedule (e.g., night-work schedule of sleep during the day and wake activities at night) and his/her internal circadian clock (i.e., endogenous circadian rhythm). Typically, Shift work sleep disorder is characterized by (daytime) insomnia and/or excessive sleepiness (during work shift hours) that occur in relation to work hours scheduled during the usual time for sleep (at least in part).

Excessive daytime sleepiness or EDS refers to persistent sleepiness at a time when the individual would be expected to be awake and alert, even during the day after apparently adequate or even prolonged nighttime sleep. EDS may be the result of a sleep disorder or a symptom of another underlying disorder such as idiopathic hypersomnia, multiple sclerosis, atypical depression, or drug-associated excessive sleepiness. While the name includes “daytime,” it is understood that the sleepiness may occur at other times that the subject should be awake, such as nighttime or other times, e.g., if the subject is working nightshift. It is also understood that EDS is medically distinct from fatigue and disorders associated with fatigue.

Attention Deficit Disorder and Attention Deficit Hyperactivity Disorder (ADD/ADHD) is a condition where a patient has difficulty controlling his or her behavior, pay attention, and attend to tasks. The principal characteristics of ADD/ADHD are inattention, hyperactivity, and impulsivity. Different symptoms may appear in different settings, depending on the demands the situation may pose for the patient's self-control.

Schizophrenia is a group of severe emotional disorders characterized by misinterpretation and retreat from reality, delusions, hallucinations, inappropriate emotional affect, and withdrawn, bizarre or regressive behavior. Several aspects of schizophrenia overlap with narcolepsy. Narcolepsy and schizophrenia may co-exist in patients. The characteristic hallucinations of schizophrenia can resemble the hypnagogic hallucinations of narcolepsy. Age of onset is similar in narcolepsy and schizophrenia, typically in the second or third decade for both diseases. Both diseases are characterized by degenerative changes in the limbic system, a region heavily innervated by hypocretin neurons. REM sleep at sleep onset is also characteristic of both disorders. Finally, disrupted nighttime sleep and daytime sleepiness can be characteristic of schizophrenia, as in narcolepsy.

Depression is characterized by a pervasive feeling of sadness or helplessness, suicidal impulses, and a loss of interest in previously pleasurable activities. It is also frequently characterized by daytime sleepiness, short sleep latency and disrupted nighttime sleep. A shortened latency to REM sleep is characteristic as is the case in narcolepsy. These sleep disturbances are strikingly similar to those seen in narcolepsy.

Anxiety refers to an uncomfortable and unjustified sense of apprehension that may be diffuse and unfocused and is often accompanied by physiological symptoms.

Psychosis refers to a clinical state characterized by delusions (false beliefs) and/or hallucinations (sensory misperceptions). The more common of these disorders recognized by the American Psychiatric Association's Diagnostic and Statistical manual of Mental Disorders (DSM-IVTR) include bipolar disorder and schizophrenia. Bipolar disorder, also known as manic-depressive illness, is manifested by recurrent episodes of mania/hypomania, depression, or a combination of both (mixed episode). Each of these stages may manifest in psychosis or give rise to a risk for the emergence of psychosis. Schizophrenia is comprised of psychotic manifestations, often depressive elements, and disruption of the basic elements of an individual's personality structure. This syndrome typically lasts over a more protracted period of time than the classic cyclic nature (recurrence) of bipolar disorder. Other psychotic disorders include: borderline personality, delusional disorder, brief reactive psychosis, schizoaffective disorder, schizophreniform disorder, psychotic major depression, psychosis due to substance abuse, and psychoses associated with medical conditions, e.g., dementia, delirium, etc.

Addiction refers to a persistent behavioral pattern marked by physical and/or psychological dependency to a substance, particularly drugs such as narcotics, stimulants, and sedatives, including but not limited to heroin, cocaine, alcohol, nicotine, caffeine, amphetamine, desoxyephedrine, methadone and combinations thereof.

Diabetes refers to a disease process characterized by elevated levels of plasma glucose or hyperglycemia. Type II diabetes, or non-insulin dependent diabetes (NIDDM or NIDD) is the form of diabetes mellitus that occurs predominantly in adults in whom adequate production of insulin is generally available for use, yet a defect exists in insulin-mediated utilization and metabolism of glucose in peripheral tissues. Signs of type II diabetes include frequent infections, blurred vision, cuts, or bruises that are slow to heal, tingling/numbness in the hands or feet, and recurring skin, gum, or bladder infections, as well as frequent urination, unusual thirst, extreme hunger, unusual weight loss, extreme fatigue, and irritability.

Metabolic syndrome is characterized by a group of metabolic risk factors present in one person. The metabolic risk factors include central obesity (excessive fat tissue in and around the abdomen), atherogenic dyslipidemia (blood fat disorders—mainly high triglycerides, low HDL/LDL ratio, and low HDL cholesterol), insulin resistance or glucose intolerance, prothrombotic state (e.g., high fibrinogen or plasminogen activator inhibitor in the blood), and high blood pressure (130/85 mmHg or higher).

Binge eating disorder is a disorder in which a patient experiences a loss of control over their eating behavior and eats notably more or differently than usual for a discrete period of time (e.g., 2 hours). Loss of control overeating may be described by the individual as feeling like they cannot stop or limit the amount or type of food eaten; having difficulty stopping eating once they have started; or giving up even trying to control their eating because they know they will end up overeating. Binge eating may be accompanied by a marked distress about the pattern of binge eating or significant impairment in personal, family, social, educational, occupational, or other important areas of functioning.

Obesity refers to a BMI between 30 and 40 in adult humans. For people under 20, obesity is defined as a BMI above the 95th percentile compared to people of the same age. As used herein, the term can include both obesity and morbid obesity.

TAAR1-associated immune disorders include systemic inflammation and autoimmunity characterized by the elevated chemokine levels or lipid mediators of inflammation, autocrine responses, or run-away immune system activation and/or an abundance of non-specifically activated T cells.

The amount of the compound necessary to provide treatment of TAAR1-associated disorders is defined as a therapeutically or a pharmaceutically effective dose. The dosage schedule and amounts effective for this use, i.e., the dosing or dosage regimen will depend on a variety of factors including the stage of the disease, the patient's physical status, age and the like. In calculating the dosage regimen for a patient, the mode of administration is also taken into account.

A person of skill in the art will be able without undue experimentation, having regard to that skill and this disclosure, to determine a therapeutically effective amount of a particular compound for practice of this invention (see, e.g., Lieberman, Pharmaceutical Dosage Forms (Vols. 1-3, 1992); Lloyd, 1999, The Art, Science and Technology of Pharmaceutical Compounding; and Pickar, 1999, Dosage Calculations). A therapeutically effective dose is also one in which any toxic or detrimental side effects of the active agent is outweighed in clinical terms by therapeutically beneficial effects. It is to be further noted that for each particular subject, specific dosage regimens should be evaluated and adjusted over time according to the individual need and professional judgment of the person administering or supervising the administration of the compounds.

For treatment purposes, the compositions or compounds disclosed herein can be administered to the subject in a single bolus delivery, via continuous delivery over an extended time period, or in a repeated administration protocol (e.g., by an hourly, daily or weekly, repeated administration protocol). The pharmaceutical formulations of the present invention can be administered, for example, one or more times daily, 3 times per week, or weekly. In one embodiment of the present invention, the pharmaceutical formulations of the present invention are orally administered once or twice daily.

In this context, a therapeutically effective dosage of the biologically active agent(s) can include repeated doses within a prolonged treatment regimen that will yield clinically significant results to provide treatment for TAAR1-associated disorders. Determination of effective dosages in this context is typically based on animal model studies followed up by human clinical trials and is guided by determining effective dosages and administration protocols that significantly reduce the occurrence or severity of targeted exposure symptoms or conditions in the subject. Suitable models in this regard include, for example, murine, rat, porcine, feline, non-human primate, and other accepted animal model subjects known in the art. Alternatively, effective dosages can be determined using in vitro models (e.g., immunologic and histopathologic assays).

Using such models, only ordinary calculations and adjustments are typically required to determine an appropriate concentration and dose to administer a therapeutically effective amount of the biologically active agent(s) (e.g., amounts that are orally effective intranasally effective, transdermally effective, intravenously effective, or intramuscularly effective to elicit a desired response). The effective amount, however, may be varied depending upon the particular compound used, the mode of administration, the strength of the preparation, the mode of administration, and the advancement of the disease condition. In addition, factors associated with the particular patient being treated, including patient age, weight, diet, and time of administration, will result in the need to adjust dosages.

In an exemplary embodiment of the present invention, unit dosage forms of the compounds are prepared for standard administration regimens. In this way, the composition can be subdivided readily into smaller doses at the physician's direction. For example, unit dosages can be made up in packeted powders, vials or ampoules and preferably in capsule or tablet form.

Effective administration of the compounds of this invention can be, for example, at an oral or parenteral dose of from about 0.01 mg/kg/dose to about 150 mg/kg/dose. For example, administration can be from about 0.1/mg/kg/dose to about 25 mg/kg/dose, e.g., from about 0.2 mg/kg/dose to about 18 mg/kg/dose, e.g., from about 0.5 mg/kg/dose to about 10 mg/kg/dose. Therefore, the therapeutically effective amount of the active ingredient can be, for example, from about 1 mg/day to about 7000 mg/day for a subject having, for example, an average weight of 70 kg, e.g., from about 10 mg/day to about 2000 mg/day, e.g., from about 50 mg/day to about 600 mg/day, e.g., about 10, 25, 37.5, 50, 75, 100, 125, 150, 175, 200, 225, 250, 275, 300, 325, 350, 375, 400, 425, 450, 475, 500, 525, 550, 575, or 600 mg/day or more or any range therein. In one embodiment, the compound of Formula I is administered in the form of a tablet or capsule at a dose of about 37.5 mg to about 300 mg without any excipients.

The methods of this invention also provide for kits for use in providing treatment or prevention of a TAAR1-associated disorder. After a pharmaceutical composition comprising one or more compounds of this invention, with the possible addition of one or more other compounds of therapeutic benefit, has been formulated in a suitable carrier, it can be placed in an appropriate container and labeled for providing treatment or prevention of a TAAR1-associated disorder. Additionally, another pharmaceutical comprising at least one other therapeutic agent can be placed in the container as well and labeled for treatment of the indicated disease. Such labeling can include, for example, instructions concerning the amount, frequency and method of administration of each pharmaceutical.

Having described the present invention, the same will be explained in greater detail in the following examples, which are included herein for illustration purposes only, and which are not intended to be limiting to the invention.

### Example 1

Solriamfetol is a wake-promoting agent (WPA) approved for the treatment of excessive daytime sleepiness associated with narcolepsy (75-150 mg/day) and obstructive sleep apnea (OSA; 37.5-150 mg/day in the US and EU. The wake promoting mechanism of solriamfetol may result from dopamine and norepinephrine reuptake inhibition (DNRI). While DNRI activity has been established for solriamfetol, its additional molecular targets are not fully characterized. In vitro and animal experiments were performed to understand the molecular targets and effects of solriamfetol in the context of other WPAs and stimulants.

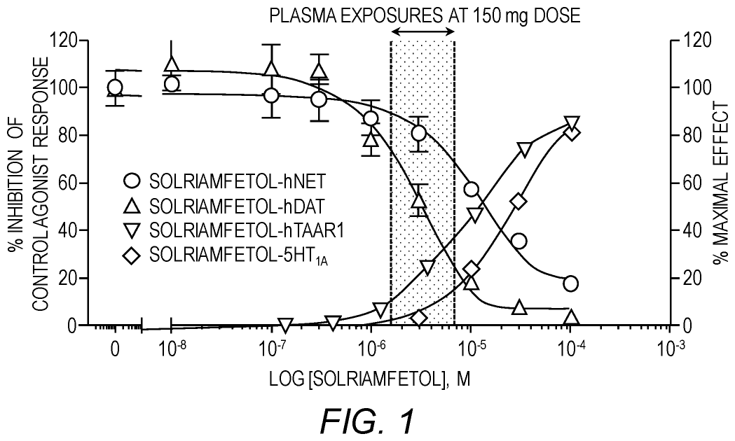

In vitro binding and functional studies were conducted in a panel of cell lines or membrane preparations expressing transmembrane receptors and monoamine transporters including human dopamine and norepinephrine transporters (hDAT, hNET, respectively), human trace amine-associated receptor 1 (hTAAR1), and serotonin 1A receptor (5-HT_{1A}) to measure the activity of solriamfetol and comparator WPAs and DNRIs. Data for stimulants (e.g., amphetamine, methamphetamine) were obtained from published literature. The results of this analysis (FIG. 1) indicated that solriamfetol is a DNRI that activates hTAAR1 and 5-HT_{1A} in vitro at clinically relevant plasma concentrations. In addition, solriamfetol and stimulants bad TAAR1 activity while modafinil did not and solriamfetol had 5-HT_{1A} activity at lower potency (Table 1). However, no additional targets were identified for solriamfetol in a binding assay panel.

TABLE 1

| | | | | |
|---|---|---|---|---|
| | hDAT | hNET | hTAAR1 | 5-HT_{1A} |
| Drug | IC₅₀ μM | IC₅₀ μM | EC₅₀ μM (Emax) | IC₅₀ μM |

WPA or hDAT/hNET inhibitor

| | | | | |
|---|---|---|---|---|
| Solriamfetol | 3.21 | 14.4 | 10-16 (100%) | 25 |
| Modafinil | 2.8 | >100 | No dose | Unknown |
| | | | response^{a} | |
| Bupropion | 0.26 | 2.79 | No dose | No functional |
| | | | response^{a} | activity |

Stimulants

| | | | | |
|---|---|---|---|---|
| (+)Amphetamine^{b} | 0.041 | 0.023 | 2.8 (91%) | Unknown |
| (+)Methamphetamine^{b} | 0.082 | 0.0013 | 5.3 (70%) | Unknown |

5-HT_{1A}, serotonin 1A receptor; EC₅₀, half maximal effective concentration; Emax, maximal effect; hDAT, human dopamine transporter; hNET, human norepinephrine transporter; hTAAR1, human trace amine-associated receptor 1; IC₅₀, half maximal inhibitory concentration; WPA, wake-promoting agent.
^{a}Data based on current studies and confirmed by published literature (Eshleman, J. Pharmacol. Exp. Ther. 289(2): 877-85 (1999))
^{b}Data from published literature (Eshleman, J. Pharmacol. Exp. Ther. 289(2): 877-85 (1999); Simmler, J. Pharmacol. Exp. Ther. 357(1): 134-44 (2016)).

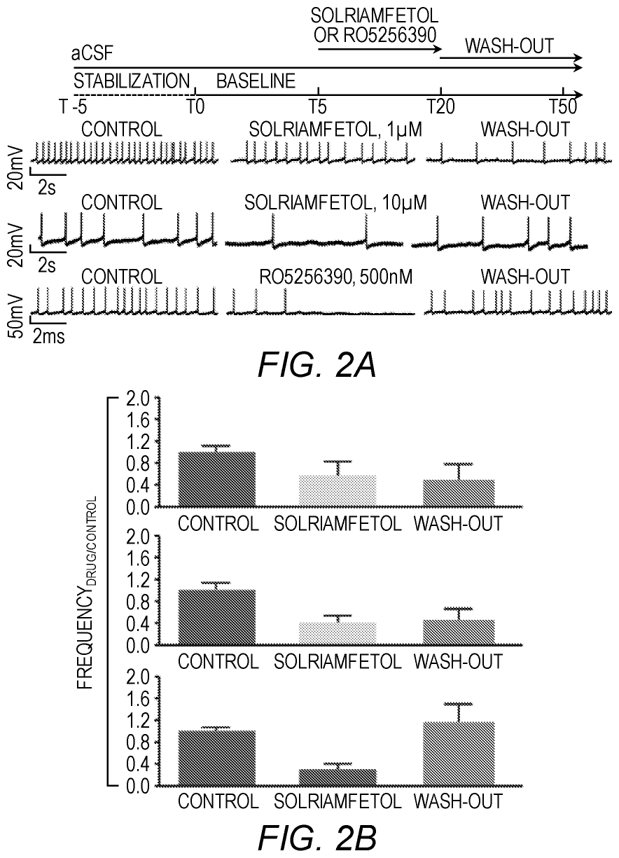

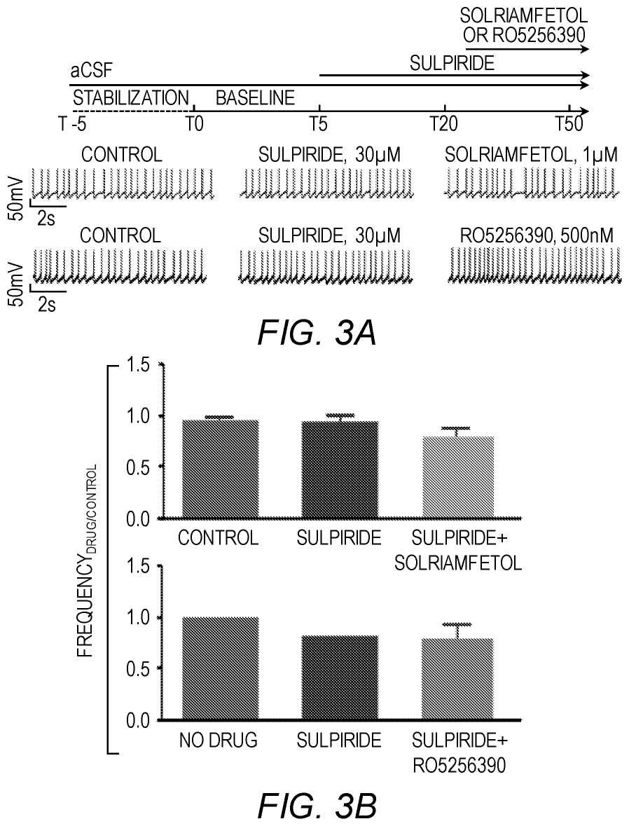

The firing frequency of ventral tegmental area (VTA) dopaminergic neurons (n=4-8 cells/experiment) in acute slice preparations was recorded using electrophysiology and analyzed. Brain slices (250 μm thickness) containing VTA from male C57Bl6/J mice were prepared using standard procedures. Slices were perfused with artificial cerebrospinal fluid (aCSF) and spontaneous action potentials were recorded from dopaminergic neurons in current clamp conditions using Axopatch 700B and pClamp10 (Axon Instruments; Revel, Proc. Natl. Acad. Sci. USA 108(20):8485-90 (2011)). The results of this analysis indicated that solriamfetol inhibited firing frequency by VTA neurons in a dose-dependent manner, similar to TAAR1 agonist ROS256390 (FIG. 2A-2B). In addition, the reduction in firing frequency by solriamfetol or TAAR1 agonist RO5256390 was antagonized by pre-treatment with the D2 receptor antagonist sulpiride (FIG. 3A-3B).

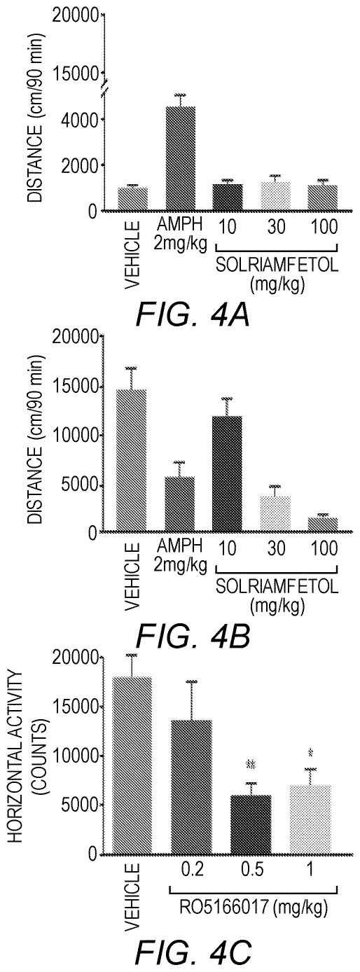

Open field locomotor activity was assessed using an automated Omnitech Digiscan (AccuScan Instruments, Columbus, OH). Wild type and DAT/mice (n=10/genotype/treatment group) received subcutaneous injections of vehicle or amphetamine (2 mg/kg) followed by solriamfetol (10 mg/kg, 30 mg/kg, or 100 mg/kg); total distance traveled (cm traveled in 90 minutes) was recorded. This analysis indicated that sofriamfetol did not increase locomotor activity in wild type mice, unlike the stimulant amphetamine (FIG. 4A). In contrast, solriamfetol reduced hyperlocomotion in DAT/mice, similar to amphetamine (FIG. 4B). Likewise, ROS166017 reduced hyperlocomotion in DAT/mice (FIG. 4C).

The results demonstrate that solriamfetol activates hTAAR1, a recently recognized component of the endogenous wake-promoting system, in vitro at potencies that are within the clinically relevant plasma concentration range and overlap with observed dopamine transporter/norepinephrine transporter inhibitory potencies. No hTAAR1 activity was observed for the WPA modafinil or the DNRI bupropion. Solriamfetol showed agonist activity at the TAAR1 receptor and a lower agonist potency at 5-HT_{1A}. This activity, in addition to its established activity as a DNRI, may contribute to the wake-promoting effects of solriamfetol. Similar to known TAAR1 agonists, solriamfetol reduced the firing frequency of mouse ventral tegmental area dopamine neurons in a D2-sensitive matter. Unlike amphetamine, solriamfetol did not promote hyperlocomotion in naïve mice; hyperlocomotion in DAT^{-/-} mice was dose-dependently inhibited by solriamfetol, similar to amphetamine.

All publications, patent applications, patents and other references cited herein are incorporated by reference in their entireties for the teachings relevant to the sentence and/or paragraph in which the reference is presented and for any other purpose for which it can be used.

1-63. (canceled)

64. A method for treating a trace amine-associated receptor 1 (TAAR1)-associated disorder selected from a sleep disorder, narcolepsy, cataplexy, idiopathic hypersomnia, shift work sleep disorder, excessive daytime sleepiness, attention deficit hyperactivity disorder, schizophrenia, depression, anxiety, psychosis, addiction, glucose homeostasis disorder, type II diabetes, metabolic syndrome, binge eating disorder, obesity and immune disorders in a subject in need thereof, comprising administering to the subject a therapeutically effective amount of a carbamoyl phenylalaninol compound which is a compound of Formula I: 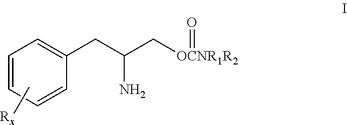
or a pharmaceutically acceptable salt or ester thereof;
wherein R is a member selected from the group consisting of alkyl of 1 to 8 carbon atoms, halogen, alkoxy of 1 to 3 carbon atoms, nitro, hydroxy, trifluoromethyl, and thioalkoxy of 1 to 3 carbon atoms;
x is an integer of 0 to 3, with the proviso that R may be the same or different when x is 2 or 3;
R₁ and R₂ are independently selected from the group consisting of hydrogen, alkyl of 1 to 8 carbon atoms, aryl, arylalkyl, cycloalkyl of 3 to 7 carbon atoms; or
R₁ and R₂ can be joined to form a 5- to 7-membered heterocycle that is unsubstituted or substituted with one or more alkyl or aryl groups, wherein the heterocycle can comprise 1 to 2 nitrogen atoms and 0 to 1 oxygen atom, wherein the nitrogen atoms are not directly connected with each other or with the oxygen atom, wherein such compound activates TAAR1, thereby treating the TAAR1-associated disorder.

65. The method of claim 64, wherein the compound of Formula I is an enantiomer of Formula I substantially free of other enantiomers or an enantiomeric mixture wherein one enantiomer of Formula I predominates.

66. The method of claim 65, wherein the enantiomer of Formula I is an enantiomer of Formula Ia: 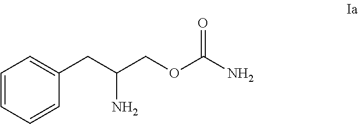 [SMILES: NC(=O)OCC(N)Cc1ccccc1]
or a pharmaceutically acceptable salt or ester thereof.

67. The method of claim 64, wherein the enantiomer of Formula I substantially free of other enantiomers is the compound of Formula Ib or an enantiomeric mixture wherein the compound of Formula Ib predominates: 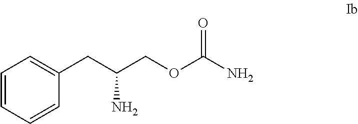 [SMILES: NC(=O)OC[C@H](N)Cc1ccccc1]
or a pharmaceutically acceptable salt or ester thereof.

68. A method for treating a trace amine-associated receptor 1 (TAAR1)-associated disorder selected from a sleep disorder, narcolepsy, cataplexy, idiopathic hypersomnia, shift work sleep disorder, excessive daytime sleepiness, attention deficit hyperactivity disorder, schizophrenia, depression, anxiety, psychosis, addiction, glucose homeostasis disorder, type II diabetes, metabolic syndrome, binge eating disorder, obesity and immune disorders in a subject in need thereof, comprising administering to the subject a therapeutically effective amount of a carbamoyl phenylalaninol compound which is a compound of Formula II: 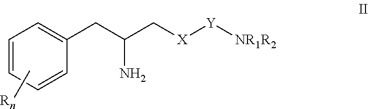
or a pharmaceutically acceptable salt thereof, wherein:
X is CH₂, O, NH, or S;
Y is C═O, C═S, or SO₂;
R is optionally substituted C₁₋₈ alkyl, halogen, optionally substituted C₁₋₄ alkoxy, cyano, hydroxy, optionally substituted trifluoromethyl, or C₁₋₄ thioalkoxy;
n is 0, 1, 2, or 3, with the proviso that R may be the same or different when x is 2 or 3; and
R₁ and R₂ can be the same or different and are independently selected from the group consisting of hydrogen, optionally substituted C₁₋₈ alkyl, optionally substituted amide, optionally substituted aryl, optionally substituted heteroaryl, optionally substituted arylalkyl, and optionally substituted C₃₋₇ cycloalkyl;
or R₁ and R₂ can be joined to form a 5- to 7-membered heterocycle optionally substituted with alkyl or aryl groups, wherein the cyclic compound can comprise 1 to 2 nitrogen atoms and 0 to 1 oxygen atom, wherein the nitrogen atoms are not directly connected with each other or with the oxygen atom;
wherein when Y is C═O, X is not O, wherein such compound activates TAAR1, thereby treating the TAAR1-associated disorder.

69. The method of claim 68, wherein the compound is a compound of Formula IIa: 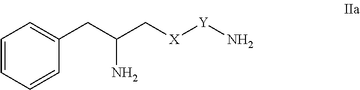
or a pharmaceutically acceptable salt thereof.

70. The method of claim 68, wherein the compound is a compound of Formula III: 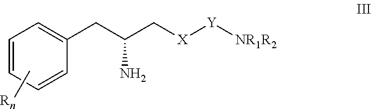
or a pharmaceutically acceptable salt thereof.

71. The method of claim 70, wherein the compound is a compound of Formula Illa: 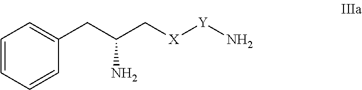
or a pharmaceutically acceptable salt thereof.

72. The method of claim 68, wherein the compound is a compound of Formula IV: 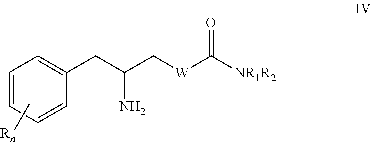
or a pharmaceutically acceptable salt thereof, wherein:
W is CH₂ or NH.

73. The method of claim 72, wherein the compound is a compound of Formula IVa: 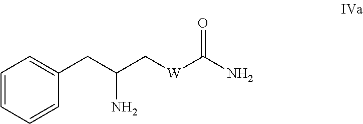
or a pharmaceutically acceptable salt thereof.

74. The method of claim 72, wherein the compound is compound 1 or 2: 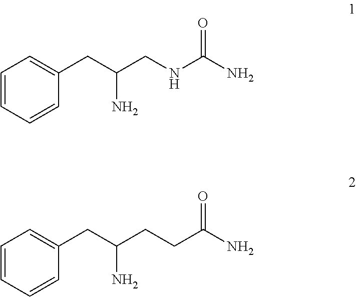 [SMILES: NC(=O)NCC(N)Cc1ccccc1 ; NC(=O)CCC(N)Cc1ccccc1]
or a pharmaceutically acceptable salt thereof.

75. The method of claim 72, wherein the compound is a compound of Formula V: 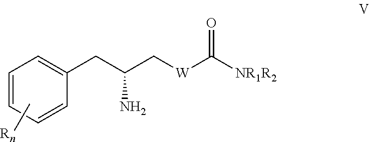
or a pharmaceutically acceptable salt thereof, wherein:
W is CH₂ or NH.

76. The method of claim 75, wherein the compound is a compound of Formula Va: 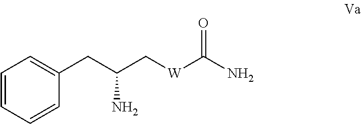
or a pharmaceutically acceptable salt thereof.

77. The method of claim 76, wherein the compound is compound 3 or 4: 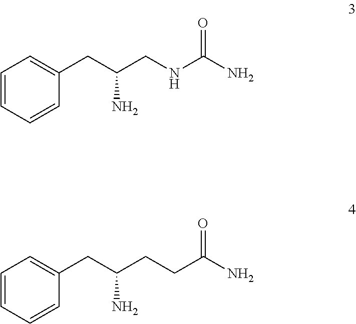 [SMILES: NC(=O)NC[C@H](N)Cc1ccccc1 ; NC(=O)CC[C@H](N)Cc1ccccc1]
or a pharmaceutically acceptable salt thereof.

78. The method of claim 68, wherein the compound is a compound of Formula VI: 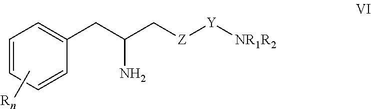
or a pharmaceutically acceptable salt thereof, wherein:
Z is O or S; and
Y is C═O, C═S, or SO₂;
wherein when Y is C═O, Z is not O.

79. The method of claim 78, wherein the compound is a compound of Formula VIa: 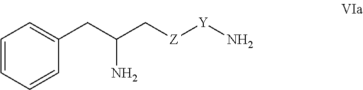
or a pharmaceutically acceptable salt thereof.

80. The method of claim 79, wherein the compound is compound 5, 6, or 7: 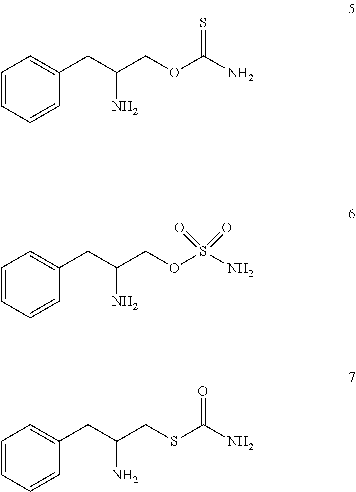 [SMILES: NC(=S)OCC(N)Cc1ccccc1 ; NC(COS(N)(=O)=O)Cc1ccccc1 ; NC(=O)SCC(N)Cc1ccccc1]
or a pharmaceutically acceptable salt thereof.

81. The compound of claim 78, wherein the compound is a compound of Formula VII: 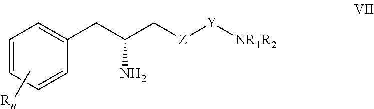
or a pharmaceutically acceptable salt thereof.

82. The compound of claim 81, wherein the compound is a compound of Formula VIIa: 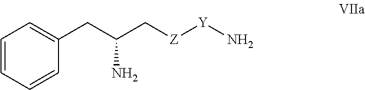
or a pharmaceutically acceptable salt thereof.

83. The compound of claim 82, wherein the compound is compound 8, 9, or 10: 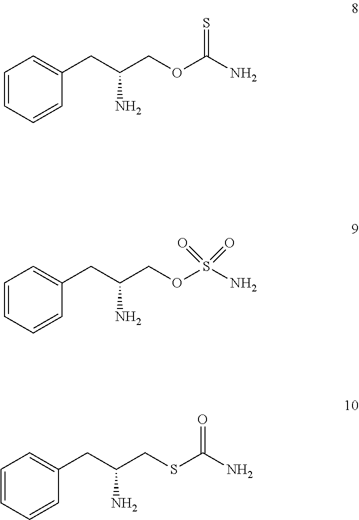 [SMILES: NC(=S)OC[C@H](N)Cc1ccccc1 ; N[C@@H](COS(N)(=O)=O)Cc1ccccc1 ; NC(=O)SC[C@H](N)Cc1ccccc1]
or a pharmaceutically acceptable salt thereof.
